(19) United States
(12) Patent Application Publication
(10) Pub. No.: US 2014/0348941 A1
(43) Pub. Date: Nov. 27, 2014

(54) METHOD FOR MANUFACTURING BIOACTIVE GLASS AND BIOACTIVE GLASS

(71) Applicants: Taipei Medical University (TMU), Taipei City (TW); National Taiwan University of Science and Technology, Taipei City (TW)

(72) Inventors: Shao-Ju SHIH, TAIPEI CITY (TW); Yu-Jen CHOU, TAIPEI CITY (TW); Chung-Kwei LIN, TAIPEI CITY (TW)

(73) Assignees: Taipei Medical University (TMU), Taipei City (TW); National Taiwan University of Science and Technology, Taipei City (TW)

(21) Appl. No.: 14/073,880

(22) Filed: Nov. 7, 2013

(30) Foreign Application Priority Data
May 24, 2013 (TW) 102118537

(51) Int. Cl. A61K 33/42

(52) U.S. Cl. CPC A61K 33/42
USPC 424/602

(57) ABSTRACT

A method for manufacturing bioactive glass includes the steps below. A precursor and a polar solvent are mixed to form a mixed solution, in which the precursor includes a silicon precursor, a calcium precursor and a phosphorus precursor. The mixed solution is atomized to form a mixture droplet. The mixture droplet is oxidized in an environment of 500° C. to 900° C. to form the bioactive glass.

### RELATED APPLICATIONS

This application claims priority to Taiwan Application Serial Number 102118537 filed May 24, 201 which is herein incorporated by reference.

### BACKGROUND

1. Technical Field

The present disclosure relates to a method for manufacturing bioactive glass and the bioactive glass for forming hydroxyl apatite.

2. Description of Related Art

Hench et al. have developed bioactive glass having good bioactivity in 1971. Layered hydroxyl apatite (HA, Ca₅(PO₄)₃(OH)) may be formed with time by implanting the bioactive glass in human body. The layered hydroxyl apatite easily forms chemical bonds with human skeleton, and thus can be used as tooth bone powders. Accordingly, the bioactive glass was being extensively and continuously studied.

The bioactive glass was in advance prepared by a traditional glass melting process. The large-size glass obtained after cooling should be grinded into glass powders. However, pollution may occur in the grinding process, and thus the purity of the bioactive glass cannot be effectively increased. In addition, the uniformity of the chemical components of the product is poor because the chemical components respectively exhibit different viscosity at high temperature. In view of this, researchers have developed a sol-gel method for preparing the bioactive glass. Various components are mixed in a liquid phase, and thus the uniformity of the chemical components of the product is better. Nevertheless, the sol-gel method only can be used to batch-produce the bioactive glass. In this regard, there is still a need of a method for continuously manufacturing the bioactive glass at present.

### SUMMARY

One aspect of the present disclosure provides a method for continuously manufacturing bioactive glass, which includes the steps of mixing a precursor and a polar solvent to form a mixed solution, in which the precursor includes a silicon precursor, a calcium precursor and a phosphorus precursor; atomizing the mixed solution to form a mixture droplet; and oxidizing the mixture droplet in an environment of 500° C. to 900° C. to form the bioactive glass.

Another aspect of the present disclosure provides a bioactive glass for forming hydroxyl apatite, which is manufactured by the above-mentioned method.

### BRIEF DESCRIPTION OF THE DRAWINGS

The disclosure may be more fully understood by reading the following detailed description of the embodiment, with reference made to the accompanying drawings as follows:

FIGS. 1-3 are schematic mechanisms for manufacturing mesoporous, solid and hollow bioactive glass according to various embodiments of the present disclosure;

FIGS. 4A-4B are TEM images of mesoporous bioactive glass according to Comparative Example 1 of the present disclosure;

FIG. 5 is a XRD pattern of mesoporous bioactive glass before and after soaking Hank's balanced salt solution according to Comparative Example 1 of the present disclosure;

FIGS. 6A-6B are TEM images of esoporous bioactive glass according to Example 1 of the present disclosure;

FIG. 7 is a XRD pattern of mesoporous bioactive glass before and after soaking Hank's balanced salt solution according to Example 1 of the present disclosure;

FIG. 8 is a TEM image of solid bioactive glass according to Example 2 of the present disclosure;

FIG. 9 is a XRD pattern of solid bioactive glass before and after soaking simulated body fluid (SBF) according to Example 2 of the present disclosure;

FIG. 10 is a TEM image of mesoporous bioactive glass according to Example 3 of the present disclosure;

FIG. 11 is a XRD pattern of mesoporous bioactive glass before and after soaking SBF according to Example 3 of the present disclosure;

FIG. 12 is a TEM image of solid bioactive glass according to Example 4 of the present disclosure;

FIG. 13 is a particle size distribution diagram of solid bioactive glass according to Example 4 of the present disclosure;

FIG. 14 is a TEM image of solid bioactive glass according to Example 5 of the present disclosure;

FIG. 15 is a particle size distribution diagram of olid bioactive glass according to Example 5 of the present disclosure;

FIG. 16 is a TEM image of hollow bioactive glass according to Example 6 of the present disclosure;

FIG. 17 is a particle size distribution diagram of hollow bioactive glass according to Example 6 of the present disclosure;

FIG. 18 is a TEM image of hollow bioactive glass according to Example 7 of the present disclosure;

FIG. 19 is a particle size distribution diagram of hollow bioactive glass according to Example 7 of the present disclosure;

FIG. 20A is a XRD pattern of solid bioactive glass before and after soaking SBF according to Example 5 of the present disclosure;

FIG. 20B is a XRD pattern of hollow bioactive glass before and after soaking SBF according to Example 7 of the present disclosure;

FIG. 21 is a TEM image of solid bioactive glass according to Example 8 of the present disclosure;

FIG. 22 is a XRD pattern of solid bioactive glass before and after soaking SBF according to Example 8 of the present disclosure;

FIG. 23 is a TEM image of solid bioactive glass according to Example 9 of the present disclosure; and

FIG. 24 is a XRD pattern of solid bioactive glass before and after soaking SBF according to Example 9 of the present disclosure.

### DETAILED DESCRIPTION

The present disclosure is described by the following specific embodiments. Those with ordinary skill in the arts can readily understand the other advantages and functions of the present disclosure after reading the disclosure of this specification. The present disclosure can also be implemented with different embodiments. Various details described in this specification can be modified based on different viewpoints and applications without departing from the scope of the present disclosure.

As used herein, the singular forms “a, nan” and “the” include plural referents unless the context clearly dictates otherwise. Therefore, reference to, for example, a mixture droplet includes aspects having two or more such mixture droplets, unless the context clearly indicates otherwise.

Reference will now be made in detail to the embodiments of the present disclosure, examples of which are illustrated in the accompanying drawings. Wherever possible, the same reference numbers e used in the drawings and the description to refer to the same or like parts

An aspect of the present disclosure provides a method for continuously manufacturing bioactive glass, which includes the steps below. First, a precursor and a polar solvent are mixed to form a mixed solution. The precursor includes a silicon precursor, a calcium precursor and a phosphorus precursor. It is sure that the precursor may further include other precursors containing other elements, such as a sodium precursor. The polar solvent may be the polar solvent that can absorb ultrasonic energy and thus transform to a form of droplet. The polar solvent may be water, ethanol, acetone, formic acid, dimethylformamide (DMF) or a combination thereof, but not limited thereto.

Water is preferably used as a main solvent since the bioactive glass should be used in human body. The polar solvent is accepted as long as being soluble for the silicon precursor, the calcium precursor and the phosphorus precursor. In one embodiment, the silicon precursor is selected from the group consisiting of tetraethyl orthosilicate (TEOS), silicon tetraacetate, tetramethoxysiliane and phenyltrimethoxysilane. In one embodiment, the calcium precursor is selected from the group consisiting of calcium nitrate tetrahydrate, calcium acetate, calcium formate and calcium nitrate. In one embodiment, the phosphorus precursor is selected from the group consisiting of triethyl phosphate (TEP) and phosphoric acid.

Silica is formed by sintering the silicon precursor, which is amorphous and can be acted as a main body of the bioactive glass. If a molar ratio of the silicon precursor to sum of the calcium precursor and the phosphorus precursor is less than 1:1, an impurity crystalline phase may be easily formed due to too low silicon content. If the molar ratio of the silicon precursor to sum of the calcium precursor and the phosphorus precursor is greater than 9:1, bioactivity of the product may be seriously affected due to too low contents of the calcium and phosphorus precursors. In addition, in one embodiment, in order to enable the bioactive glass to effectively form hydroxyl apatite, a molar ratio of the calcium precursor to the phosphorus precursor is about 1:1 to about 3:1.

In one embodiment, in order to form mesoporous spherical bioactive glass, the step of mixing the precursor and the polar solvent includes mixing a copolymer surfactant with the precursor and the polar solvent. The copolymer surfactant includes ethylene oxide and propylene oxide, such as tri-block copolymer or linear copolymer. The tri-block copolymer may be Pluronic F-127 (EO₁₀₆PO₇₀EO₁₀₆) or Pluronic P-123 (EO₂₀PO₇₀EO₂₀). The linear copolymer may be Pluronic F-68 ((C₃H₆O—C₂H₄O)ₓ). When the copolymer surfactant has an amount greater than 51 wt % based on the total weight of the precursor and the copolymer surfactant, the surfactant is not easy to be removed, and thus specific surface area of the mesoporous bioactive glass particle may be significantly reduced, which results in low bioactivity. When the copolymer surfactant has an amount lower than 10 wt % based on the total weight of the precursor and the copolymer surfactant, the surfactant fails to form three-dimensional tubular (micelles) structure and is not easy to be burned and removed during heating process, which results in low bioactivity. Therefore, the copolymer surfactant preferably has an amount of about 10 to about 51 wt % based on the total weight of the precursor and the copolymer surfactant.

In the embodiment that the mixed solution including the copolymer surfactant, an acid is mixed with the precursor, the polar solvent and the copolymer surfactant. The acid, such as hydrochloric acid, is used to let the copolymer surfactant easily crosslink in the acidic environment to form micelles.

Next, the mixed solution is atomized to form mixture droplets. For an example, an ultrasonic humidifier may be used to disperse the mixed solution into droplets. In one embodiment, frequency of the ultrasonic vibration is in a range of 1.0 MHz to 3.0 MHz. If the precursor has an amount greater than 10 wt %, the mixed solution may not easy to be atomized. Accordingly, the amount of the precursor is preferably less than or equal to 10 wt % based on the total weight of the precursor and the polar solvent. If the amount of the precursor less than 0.01 wt %, solute concentration of the droplet is too low, which results in low yield (lower than or equal to 30%), long process time (about four times or more) and high process cost. Given the above, the amount of the precursor is preferably in a range of 0.01 to 10 wt % based on the total weight of the precursor and the polar solvent.

Hereinafter, the mixture droplets are oxidized in an environment of 500° C. to 900° C. to form the bioactive glass. In one embodiment, the mixture droplet has a diameter in a range of about 1 to about 10 μm, and the bioactive glass has a diameter in a range of about 10 nm to about 3.0 μm. Specifically, the mixture droplets are continuously fed to a tubular furnace with the temperature setting at 500° C. to 900° C. by an air stream generated by a suction pump, and the bioactive glass is then formed and collected at the end of the tubular furnace. As such, the embodiment of the present disclosure can be applied in a continuous manufacturing process to mass-produce the bioactive glass. In one embodiment, process time of oxidizing the mixture droplet in the above-mentioned environment is less than or equal to about 1 minute, such that process time can be significantly reduced. In contrast to the sol-gel method, the present process can be efficiently increased.

After the step of oxidizing, the bioactive glass is cooled to form the amorphous bioactive glass. Specifically, a temperature-controlled type tubular furnace capable of setting a number of temperature sections may be used. For an example, there may be a preheating section, a calcining section and a cooling section, and the calcining section may be set at 500° C. to 900° C. The set temperature of the preheating section should be lower than that of the calcining section, such as 250° C. to 500° C. The set temperature of the cooling section should also be lower than that of the calcining section, such as 300° C. to 500° C., but the present disclosure is not limited to those disclosed a hove.

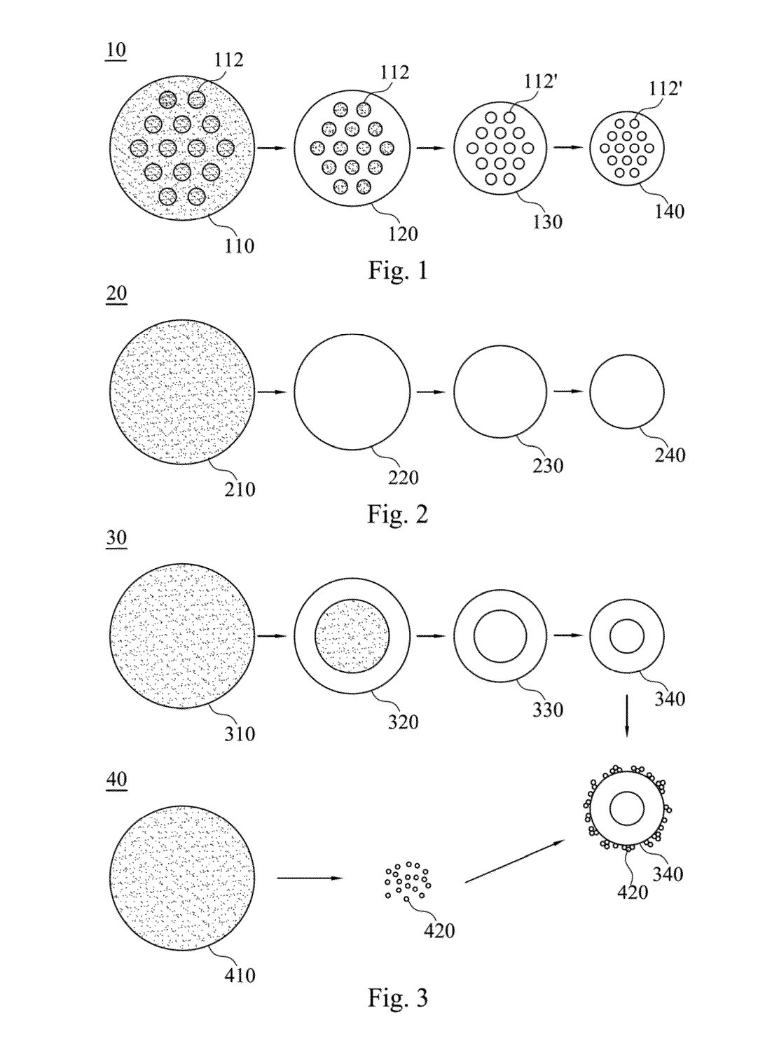

Please refer to FIG. 1, which is a formation mechanism 10 of a mesoporous spherical bioactive glass 140. First, a mixed solution, which should include a copolymer surfactant, is transformed into mixture droplets 110 by an atomization process. A number of micelles 112 are formed when reaching a critical concentration of the copolymer surfactant during the atomization process. The micelle 112 includes the copolymer surfactant and the precursor. Next, the polar solvent in the mixture droplet 110 is evaporated and thus the precursor is precipitated to form a particle 120 during a thermal process. The organic compound of the micelle 112 of the particle 120 is then thermally degraded to form a particle 130 having a plurality of pores 112′. After calcining the particle 130, the mesoporous spherical bioactive glass 140 is formed. The formation mechanism 10 belongs to a mechanism of “one-particle-per-drop”.

Please refer to FIG. 2, which is a formation mechanism 20 of a solid spherical bioactive glass 240, First, a mixed solution without any copolymer surfactant is transformed into mixture droplets 210 by an atomization process. Next, the polar solvent in the mixture droplet 210 is evaporated and thus the precursor is precipitated to form a particle 220 during a thermal process. The precursor of the particle 220 is then pyrolyzed uniformly into form a particle 230. After calcining the particle 230, the solid spherical bioactive glass 240 is formed. The formation mechanism 20 also belongs to the mechanism of “one-particle-per-drop”. In particular, the inventors have found that solid or hollow bioactive glass can be formed by using various process conditions when using a same mixed solution. The hollow bioactive glass has the advantage of low material cost, and can be used as a drug carrier. In the following examples, how to form the solid or hollow bioactive glass by using different calcining temperatures when using a same mixed solution will be described in detail below.

Please refer to FIG. 3, which is a formation mechanism 30 of a hollow spherical bioactive glass 340. First, a mixed solution without any copolymer surfactant is transformed into mixture droplets 310 by an atomization process. Next, the precursor on the surface of the mixture droplet 310 is precipitated during a thermal process, but the precursor in the mixture droplet 310 is too late to precipitate, and thus a particle 320 having a droplet therein is formed. For an instance, a high calcining temperature is used to let the external temperature of the mixture droplet be higher than the internal temperature thereof, such that the precursor located at outer portion may be firstly precipitated. The polar solvent of the droplet in the particle 320 is then evaporated to form a hollow particle 330. The particle 330 is cooled and shrank to form the hollow spherical bioactive glass 340. The formation mechanism 30 also belongs to the mechanism of “one-particle-per-drop”.

Please continuously referring to a formation mechanism 40 of FIG. 3. Some precursors of the mixture droplet 410 may have lower initial thermal degradation temperature (i.e., poor heat resistance), and thus may be directly vaporized at high calcining temperature and then condensed to form nanoparticles 420 during cooling. The mechanism 40 may be called as “gas to particle conversion”. Therefore, the hollow bioactive glass 340 with the nanoparticles 420 adhered thereon may be formed when using a high calcining temperature.

### Embodiments

### A. Mesoporous Bioactive Glass Prepared by Different Processes Comparative Example 1: Mesoporous Bioactive Glass Prepared by Sol-gel Method

7.00 g tri-block copolymer F-127 (Pluronic F-127, Sigma-Aldrich, Germany), 6.70 g TEAS (Si(OC₂H₅)₄, 99.8 wt %, Showa, Japan), 1.40 g calcium nitrate tetrahydrate (CN, Ca(NO₃)₂. 4H₂O 98.5 wt %, Showa, Japan), 0.73 g TEP ((C₂H₅)₃PO₄, 99 wt %, Alfa Aesar, USA), 1.00 g HCl (0.5 M) and 60.00 g ethanol were stirred at room temperature for 24 hours to form a sol-gel solution. The molar ratio of Si:Ca:P of the sol-gel solution was 76:14:10. The sol-gel solution was dried in an air environment for 24 hours to form a gel. The gel was heated to 700° C. with a ramp rate of 5° C./min and then calcined at 700° C. for 2 hours. Next, the product was cooled to room temperature with a cooling rate of 5° C./min, and then polished and sieved (plastic sieve, mesh size: 105 μm) to obtain the mesoporous bioactive glass prepared by the sol-gel method.

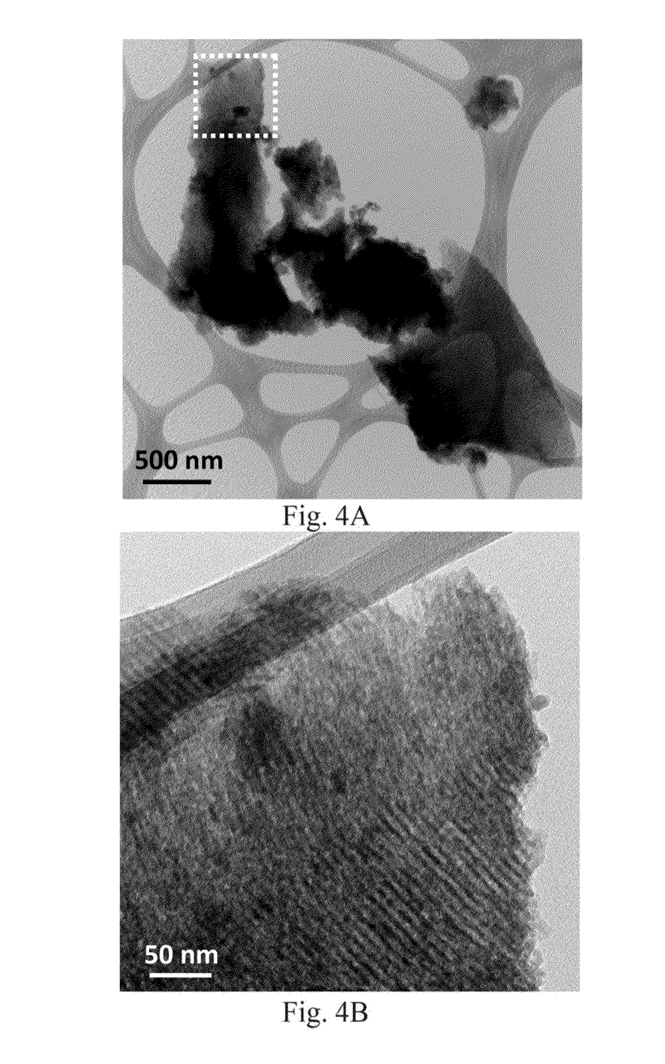

As shown in FIG. 4A, the mesoporous bioactive glass was flaky and had Edi an irregular surface. FIG. 4B is a partially enlarged view of FIG. 4A. As shown in FIG. 4B, the flaky mesoporous bioactive glass had a plurality of long tubes closely arranged. The average pore size was 5±1 nm calculated by a minimum diameter of each long tube in a cross-sectional view. In addition, the specific surface area was 193±2 m²/g tested by BET specific surface area measuring instrument. The molar ratio of Si:Ca:P of the mesoporous bioactive glass of Comparative Example 1 was 84±3:14±3:2±1 tested by X-ray Energy Dispersive Spectrometer (REDS), which was slightly different from that of the sol-gel solution.

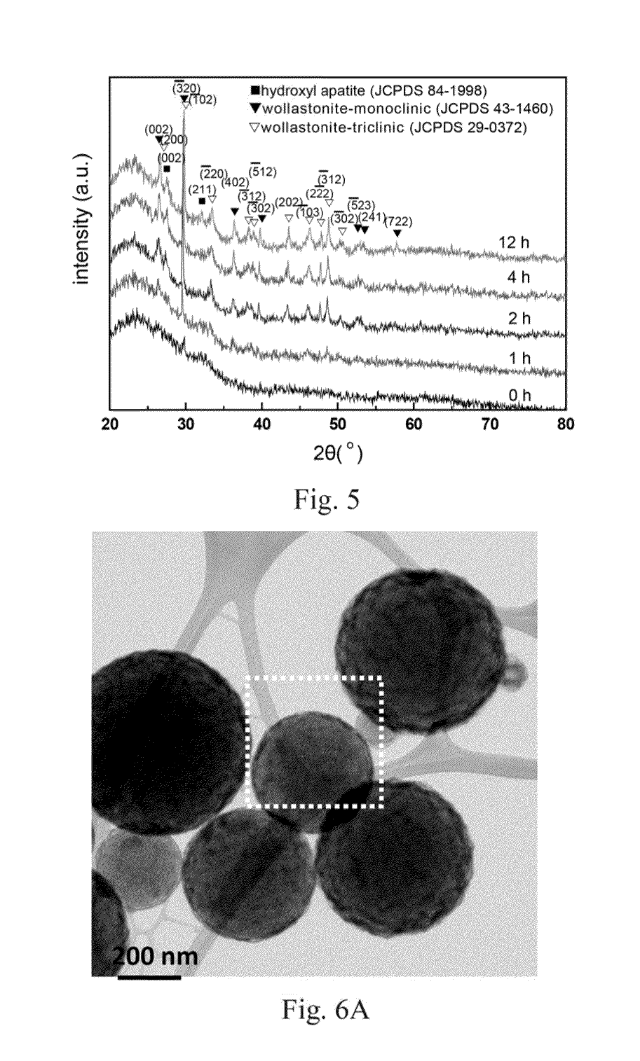

FIG. 5 is a XRD pattern of the mesoporous bioactive glass of Comparative Example 1 before (0 hr) and after soaking Hank's balanced salt solution (1, 2, 4, 12 hrs). Hank's balanced salt solution included Na⁺140.0 mmolL⁻¹(mM), K⁺ 27.0 mmolL⁻¹, Mg²⁺0.5 mmolL⁻¹, Ca²⁺0.5 mmolL⁻¹, Cl⁺169.0 mmolL⁻¹, HPO₄²⁻0.34 mmolL-1 and SO₄²⁻10 mmolL⁻¹. The product before soaking Hank's balanced salt solution had a broad band in a range of 20° to 37° and a weak peak at about 30°, which represents that the product was mainly amorphous structure and included a small amount of monoclinic wollastonite. After soaking 1 hr, triclinic wollastonite was formed. After soaking 2 hrs, two characteristic peaks of (002) and (211) were respectively formed at 25.8° and 31.7° which represents that hydroxyl apatite was formed.

### EXAMPLE 1

### Mesoporous Bioactive Glass Prepared by Process of the Present Disclosure

10 ml sal-gel solution of Comparative Example 1 and 390 ml deionized water were mixed to form a mixed solution. The molar ratio of Si:Ca:P of the mixed solution was still 76:14:10. The mixed solution was atomized by an atomizer with a frequency of 1.65 MHz to form mixture droplets. The mixture droplets were then fed into a tubular reactor, which had a preheating section, a calcining section and a cooling section set at 250, 700 and 350 ° C. in order, to perform solvent evaporation, solute precipitation, precursor degradation and oxidation reaction to form the mesoporous bioactive glass particles. Finally, the surface of the particles was charged by electron released from a tungsten corona wire at high voltage (16 kV), and the negative charged particles were then neutralized and condensed in an earthed stainless steel collector.

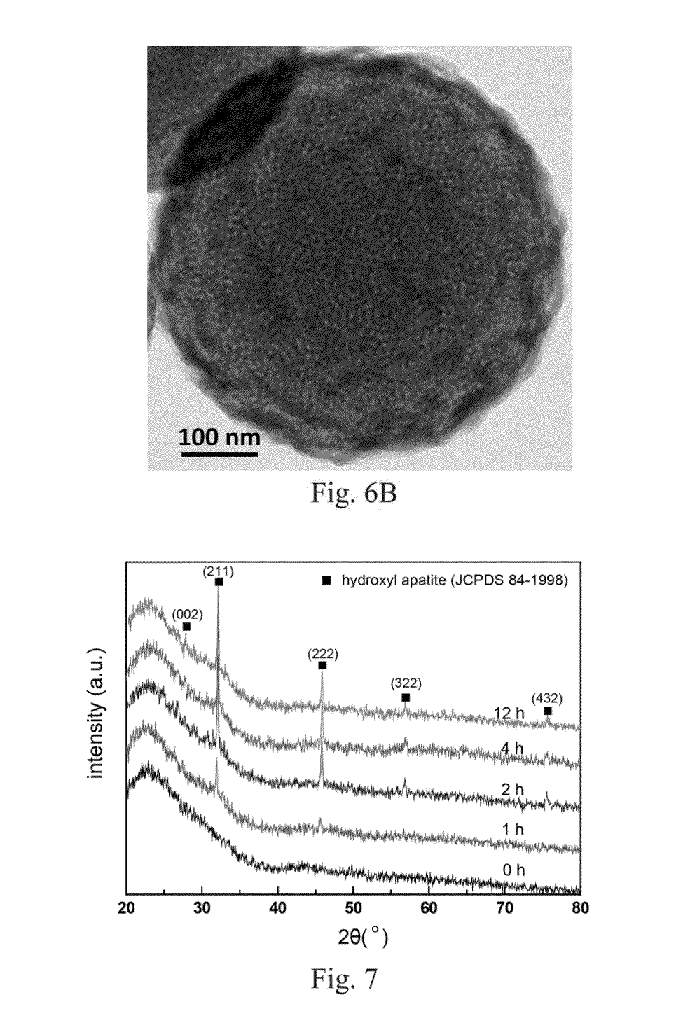

As shown in FIG. 6A, the mesoporous bioactive glass was spherical since the precursor could be precipitated uniformly (i.e., volume precipitation) and thermal degraded. As shown in FIG. 6B, the pores were uniformly distributed. The average pore size was 6±1 nm calculated by a diameter of each pore in a cross-sectional view. In addition, the specific surface area was 261±1 tested by BET specific surface area measuring instrument. The molar ratio of Si:Ca:P of the mesoporous bioactive glass of Example 1 was 72±3:17±1:11±1 tested by:REDS. Therefore, the molar ratio of Si:Ca:P of the product of Example 1 was closer to the initial mixed solution than that of Comparative Example 1. It can be seen that the process of the present disclosure can precisely control the composition of the product. As shown in FIG. 7, the product before soaking Hank's balanced salt solution did not have any crystalline phase. After soaking 1 hr, hydroxyl apatite was formed. During soaking 2 to 12 hrs, intensity of characteristic peaks of hydroxyl apatite of a single crystalline phase was continuously increased. Accordingly, the product of Example 1 had excellent bioactivity than that of Comparative Example 1, and did not have any impurity crystalline phase.

### B. Solid and Mesoporous Bioactive Glass Prepared by Process of the Present Disclosure

### EXAMPLE 2

### Solid Bioactive Glass

The difference between the composition of Example 2 and that of Comparative Example 1 was that the composition of Example 2 did not include tri-block copolymer F-127. That is, the sol-gel solution of Example 2 only included 6.70 g TEOS, 1.40 g CN, 0.73 g TEP, 1.00 g HCl (0.5 M) and 60.00 g ethanol. The process of Example 2 was similar to that of Example 1, and the difference between those was that the preheating section, the calcining section and the cooling section of Example 2 were set at 250, 700 and 350° C. in order.

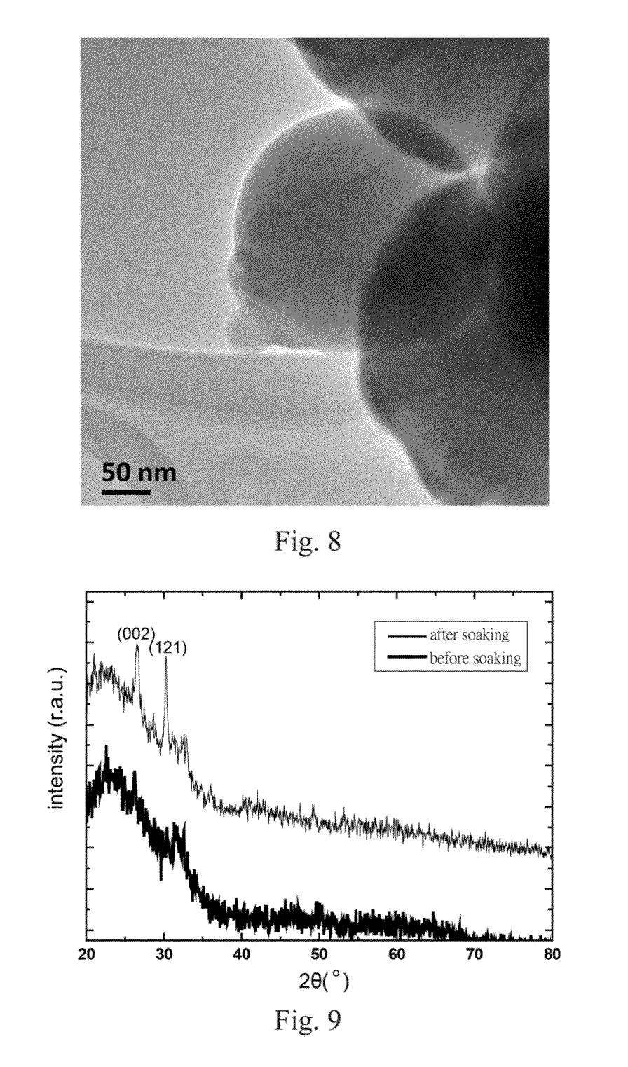

As shown in FIG. 8 the particle of Example 2 was uniformly spherical and had a diameter in a range of about 50 to 470 nm. The specific surface area was 8.0 m²/g tested by BET specific surface area measuring instrument. The molar ratio of Si:Ca:P of the solid bioactive glass of Example 2 was 74.8:15.6:9.6 tested by XEDS, which was close to that of the initial mixed solution. It can be seen that the process of the present disclosure can precisely control the compostion of the product. FIG. 9 is a XRD pattern of the solid bioactive glass of Example 2 before and after soaking simulated body fluid (SBF). The SBF was known as Kokubo solution, which included Na⁺142.0 mmolL⁻¹(mM), K⁺5.0 mmolL⁻¹, Mg²⁺1.5 mmolL⁻¹, Ca²⁺2.5 mmolL⁻¹, Cl⁻147.8 mmolL⁻¹, HCO₃⁻4.2 mmolL⁻¹, HPO₄²⁻1.0 mmolL⁻¹ and SO₄²⁻0.5 mmolL⁻¹. The product before soaking SBF only had a broad band in a range of 20° to 37° and did not have any crystalline phase. After soaking 0.5 hr, two characteristic peaks of (002) and (211) respectively at 25.8° and 31.7° were formed, which represents that hydroxyl apatite was formed. The crystal size of hydroxyl apatite was about 5 nm, which was calculated according to Scherrer's formula (Cullity and Stock 2001).

### EXAMPLE 3

### Mesoporous Bioactive Glass

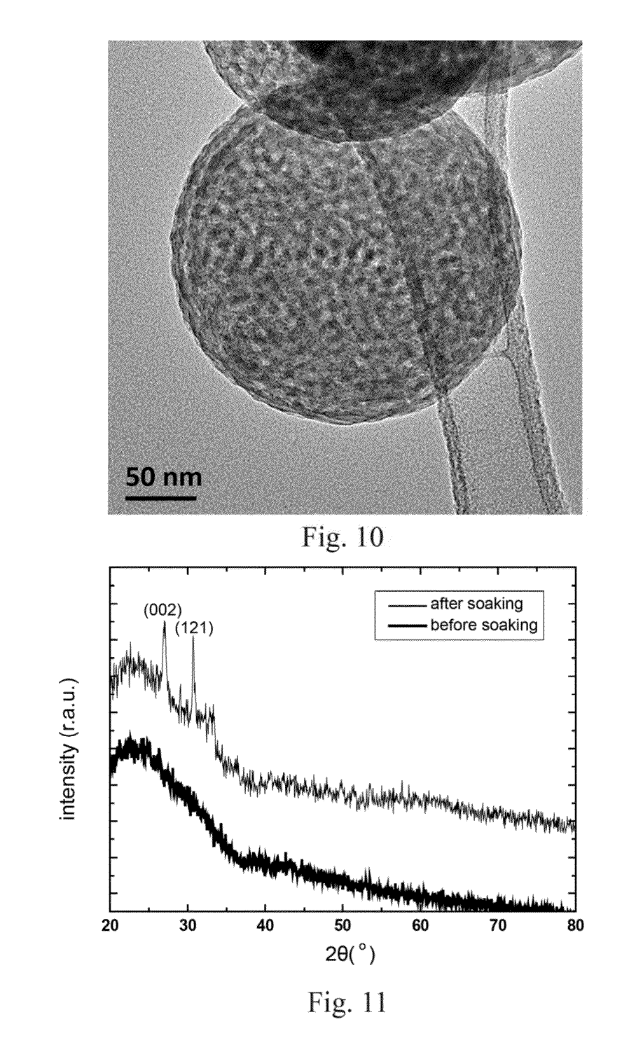

The composition and the process of Example 3 were the same as those of Example 1. As shown in FIG. 10, the particle of Example 3 was mesoporous spherical and had a diameter in a range of about 35 to 1050 nm. The specific surface area was 250.1 m²/g tested by BET specific surface area measuring instrument. The molar ratio of Si:Ca:P of the mesoporous bioactive glass of Example 3 was 77.7:12.9:9.5 tested by REDS, which was close to that of the initial mixed solution. As mentioned above, the process of the present disclosure can precisely control the composition of the product. As shown in FIG. 11, the product before soaking SBF only had a broad band at 20° to 37° and did not have any crystalline phase. After soaking 0.5 hr, characteristic peaks of (002), (211) and (222) were generated, which represents that hydroxyl apatite was formed. The crystal size of hydroxyl apatite was about 40 nm, which was calculated according to Scherrer's formula. Since the specific surface area of the particle of Example 3 was greater than that of Example 2, the bioactivity of the particle of Example 3 was better, so as to form larger-size hydroxyl apatite after soaking Hank's balanced salt solution for 0.5 hr.

### C. Bioactive Glass Prepared by Using Different Calcining Temperatures Example 4: Solid Bioactive Glass (Calcining Temperature: 500° C.)

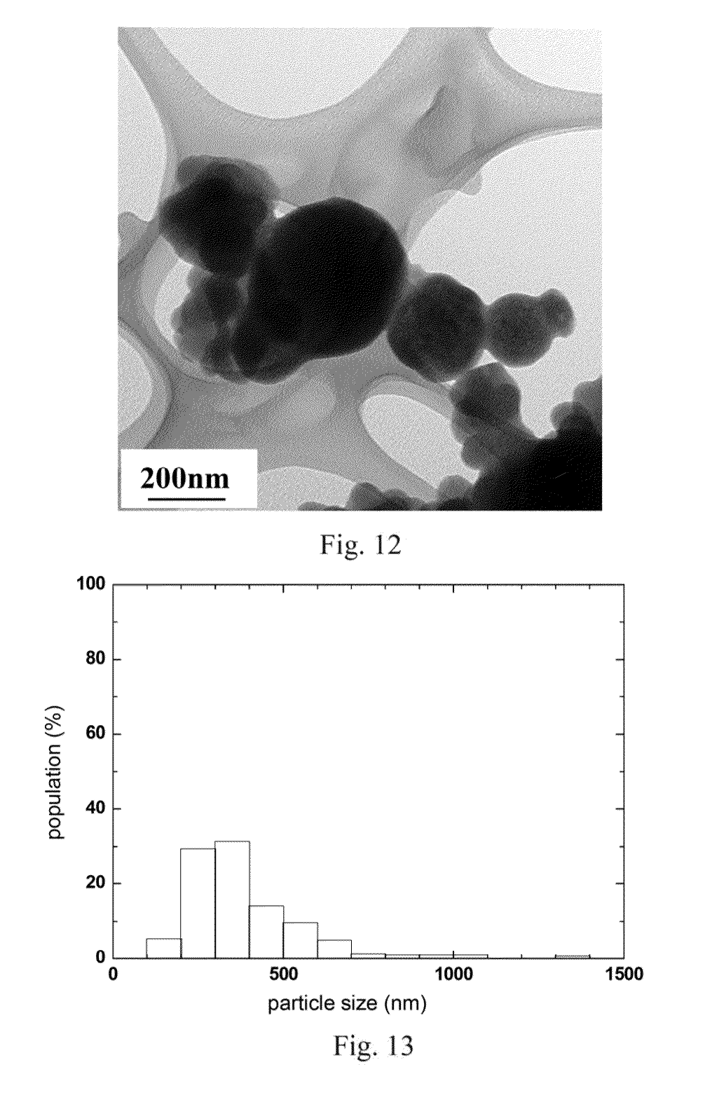

The composition of Example 4 was the same as that of Example 2, and the difference of the process was that the preheating section, the calcining section and the cooling section of the tubular reactor were set at 400, 500 and 500° C. in order. The size of the mixture droplets was 6±2 μm. As shown in FIG. 12, the product was solid bioactive glass. As shown in FIG. 13, the particle size distribution of the product belongs to a normal distribution, and an average particle size was 383±176 nm. In addition, the specific surface area was 6.0 m²/g tested by BET specific surface area measuring instrument.

### EXAMPLE 5

### Solid Bioactive Glass (Calcining Temperature: 600° C.)

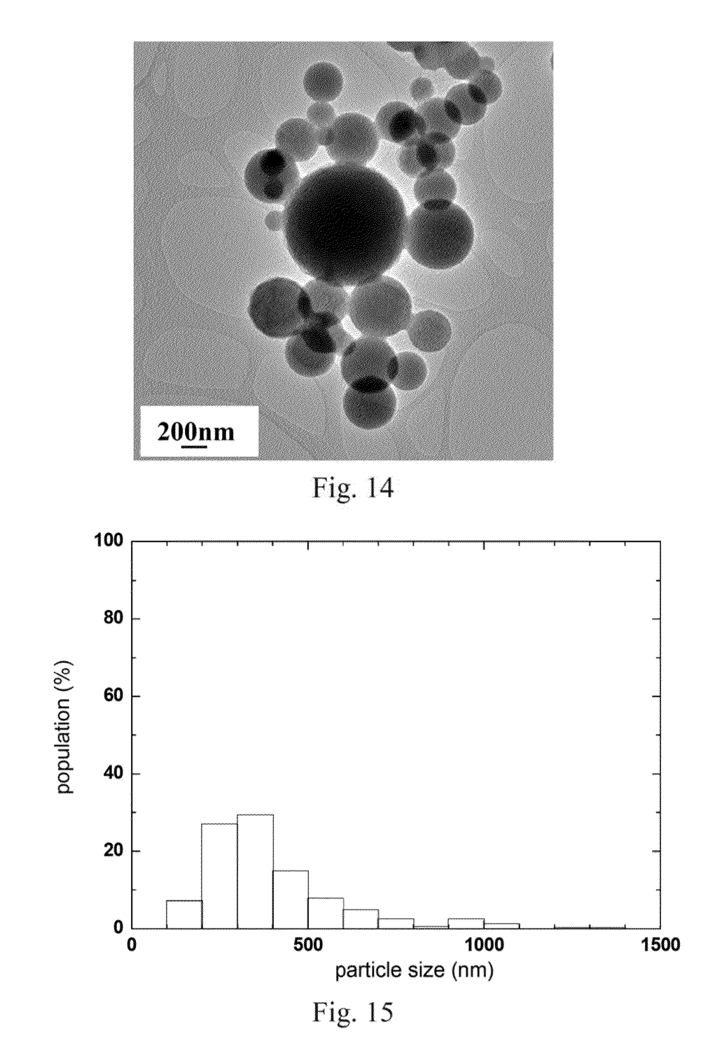

The composition of Example 5 was the same as that of Example 4, and the difference of the process was that the preheating section, the calcining section and the cooling section of the tubular reactor were set at 400, 600 and 500° C. in order. As shown in FIG. 14, the product was solid bioactive glass. As shown in FIG. 15, the particle size distribution of the product belongs to a normal distribution, and an average particle size was 404±216 nm. In addition, the specific surface area was 7.3 m²/g tested by BET specific surface area measuring instrument.

### EXAMPLE 6

### Hollow Bioactive Glass (Calcining Temperature: 700° C.)

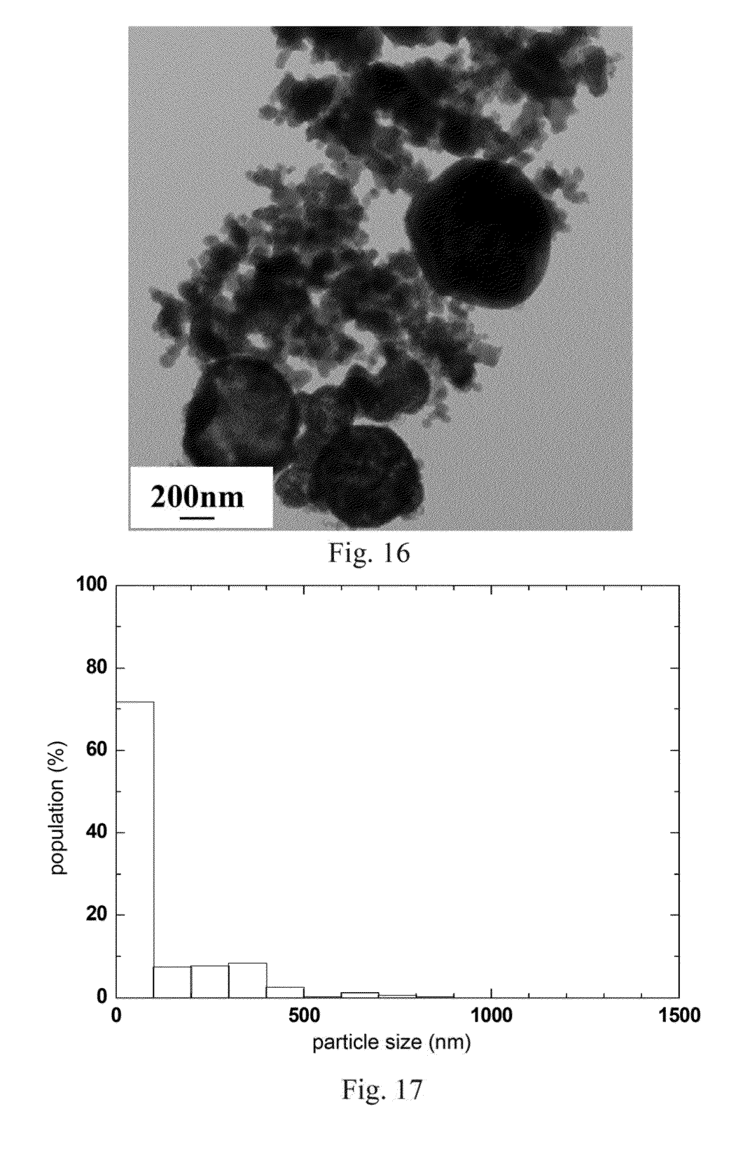

The composition of Example 6 was the same as that of Example 4, and the difference of the process was that the preheating section, the calcining section and the cooling section of the tubular reactor were set at 400, 700 and 500° C. in order. As shown in FIG. 16, distinguishing discontinuous contrast patterns were observed in the middle of particles,which indicated that the particles may be hollow spheres. As shown in FIG. 17, the particle size distribution of the product belongs to a bi-modal distribution, and an average particle size was 129±136 nm In addition, the specific surface area was 8.6 m²/g tested by BET specific surface area measuring instrument.

### EXAMPLE 7

### Hollow Bioactive Glass (Calcining Temperature: 800° C.)

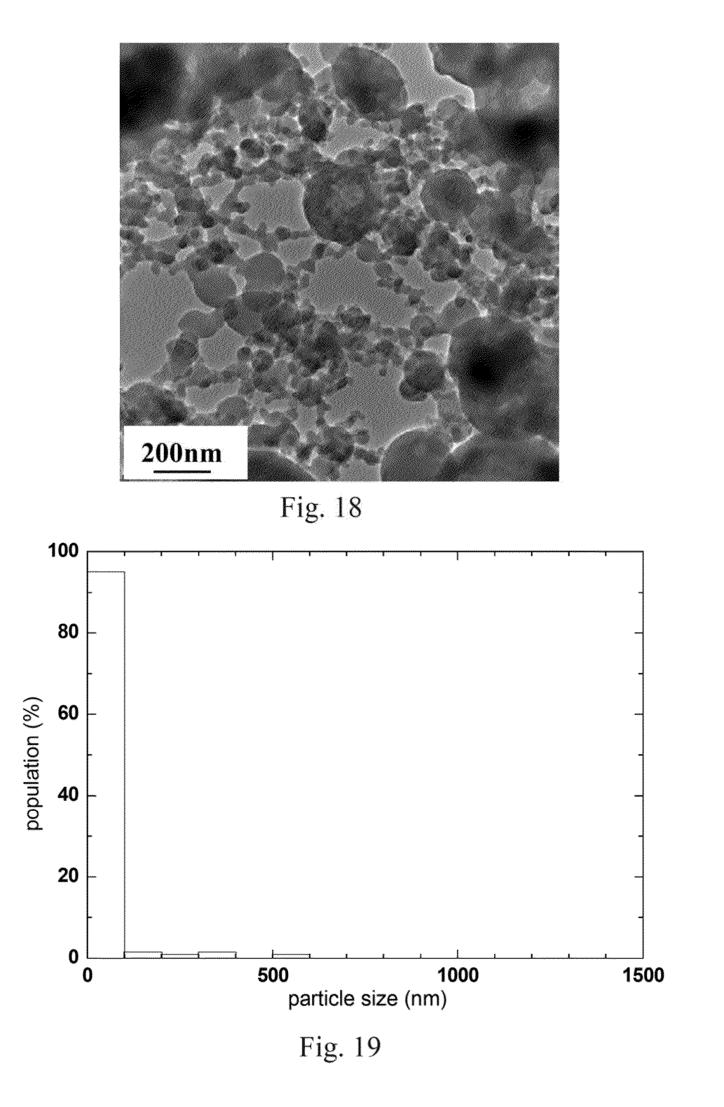

The composition of Example 7 was the same as that of Example 4, and the difference of the process s that the preheating section, the calcining section and the cooling section of the tubular reactor were set at 400, 800 and 500° C. in order. As shown in FIG. 18, distinguishing discontinuous contrast patterns were observed in the middle of particles, which indicated that the particles may be hollow spheres. As shown in FIG. 19 the particle size distribution of the product belongs to a bi-modal distribution, and an average particle size wwas 48±70 nm. In addition, the specific surface area was 20.1 m²/g tested by BET specific surface area measuring instrument.

As mentioned above, the calcining temperature may affect the formed structure of the bioactive glass. When the calcining temperature is in a range of 500 to 600° C., the precursor may be uniformly precipitated from the mixture droplet so as to form the solid spherical particle. That is, the formation mechanism is the mechanism of “one-particle-per-drop”, as shown in FIG. 2.

However, when the calcining temperature is in a range of 700 to 800° C., sub-micron particles were transformed into hollow particles. It is because the external temperature of the droplet is higher than the internal temperature at higher calcining temperature. Therefore, the precipitation reaction occurs at the surface of the droplet rather than the inside thereof, so as to form the hollow structure, as shown in FIG. 3. Further, as shown in FIGS. 16 and 18, the product included not only the particles but also the fine nanostructures distributed outside of the particles; as shown in FIGS. 17 and 19, the products included a great amount of nanoparticles with a diameter low than or equal to 100 nm. The nanoparticles may be formed by the mechanism of “gas to particle conversion”.

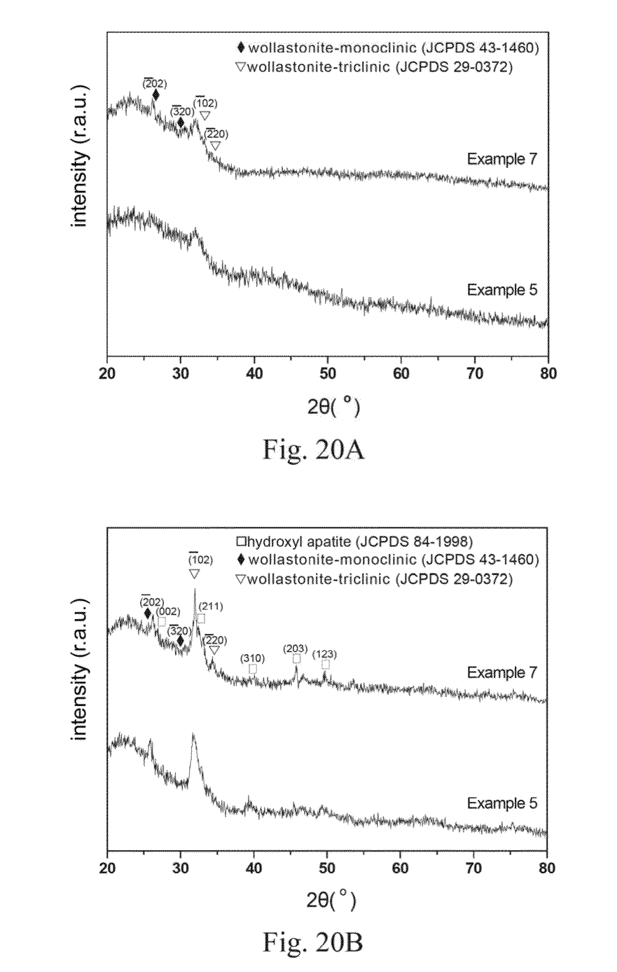

Moreover, the bioactive test was performed on the products of Examples 5 and 7, and the results are shown in FIGS. 20A-20B. As shown in FIG. 20A, the product of Example 7 before soaking SBF had monooclinic and triclinic wollastonite. As shown in FIG. 20B, two characteristic peaks of (002) and (211) respectively at 25.8° and 31.7° were generated after soaking the product of Example 7 for 1 hr, but only the characteristic peak (211) was generated after soaking the product of Example 5. Accordingly, the product of Example 7 had better bioactivity than that of Example 5 since the product of Example 7 had greater specific surface area.

### D. Solid Bioactive Glass Prepared by Different Precurors Example 8: Solid Bioactive Glass (Calcining Temperature: 600° C.)

The difference of the composition of Example 8 and that of Example 2 that the silicon precursor of Example 8 was silicon tetraacetate rather than TEOS. That is, the sol-gel solution of Example 8 included 6.70 g silicon tetraacetate, 1.40 g CN, 0.73 g TEP, 1.00 g HCl (0.5 M) and 60.00 g ethanol. The process of Example 8 was similar to that of Example 2, and the difference between those was that the preheating section, the calcining section and the cooling section were set at 250, 600 and 350° C. in order.

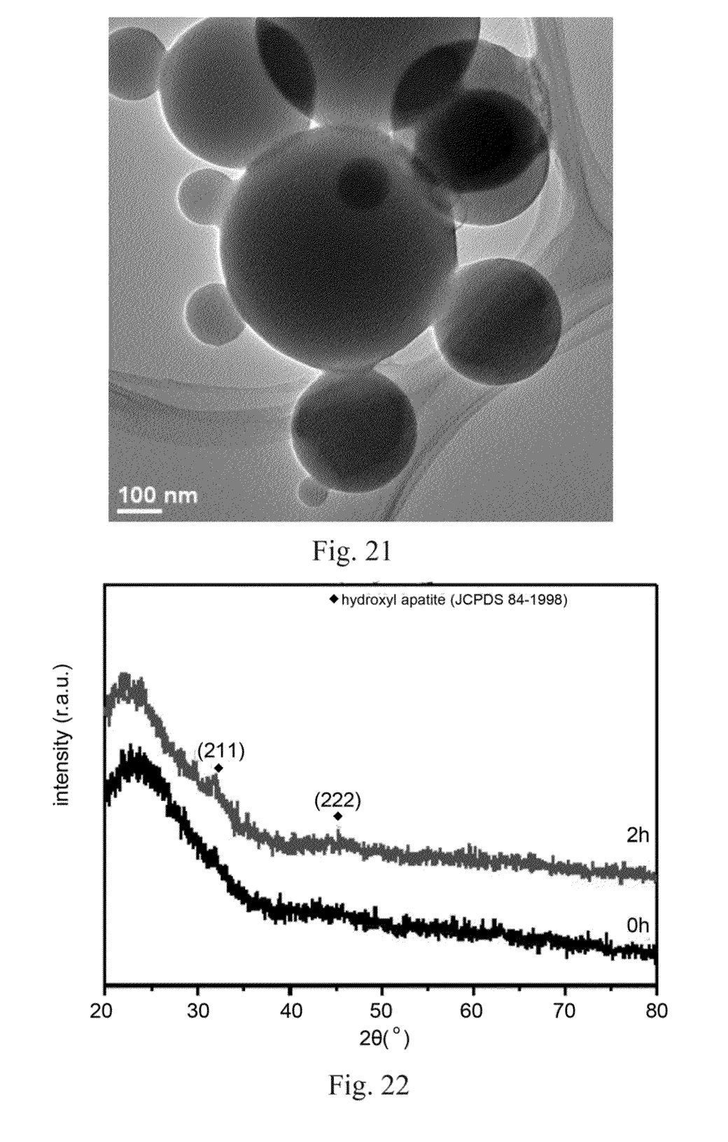

As shown in FIG. 21 the particle of Example 8 was a regular sphere. As shown in FIG. 22, the product only had a broad band in a range 20 to 37 and did not have any crystalline phase before soaking SBF. After soaking 2 hrs, two characteristic peaks of (211) and (222) were generated, which represents that hydroxyl apatite had been formed.

### EXAMPLE 9

### Solid Bioactive Glass (Calcining Temperature: 700° C.)

The composition of Example 9 was the same as that of Example 8, and the difference of the process was that the preheating section, the calcining section and the cooling section of the tubular reactor were set at 250, 700 and 350° C. in order.

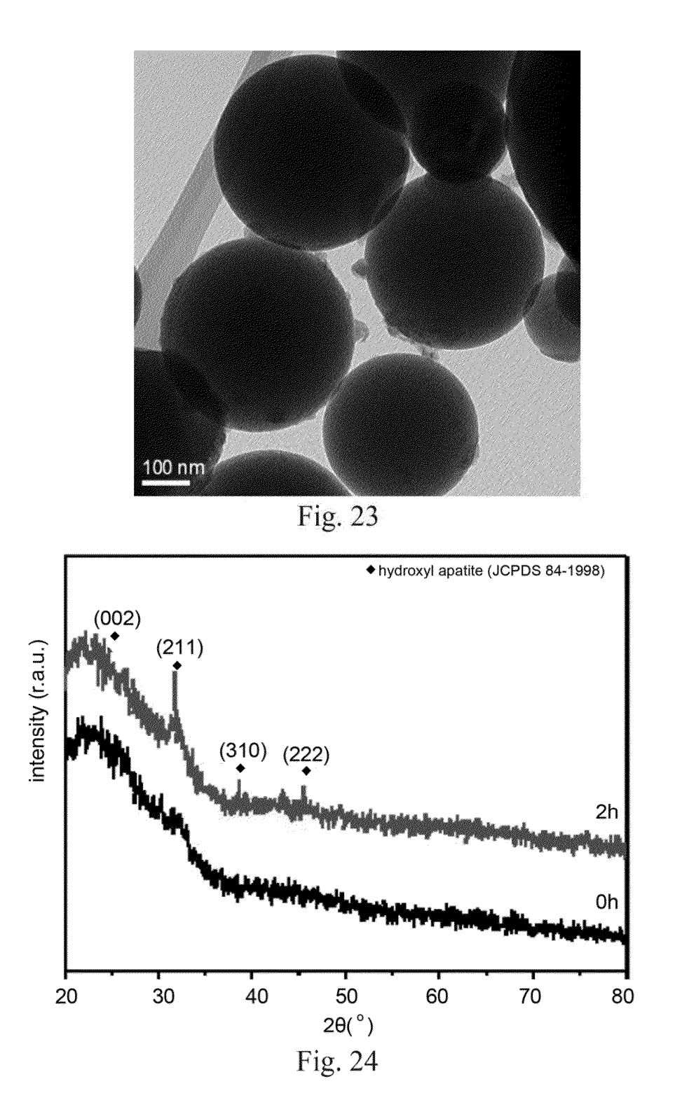

As shown in FIG. 23, the particle of Example 9 was a regular sphere. As shown in FIG. 24, the product only had a broad band in a range 20 to 37° and did not have any crystalline phase before soaking SBF. After soaking 2 hrs, four characteristic peaks of (002), (211), (310) and (222) were generated, which represents that hydroxyl apatite had been formed. Since the thermal degradation temperature of silicon tetraacetate was high at the calcining temperature of 700° C., the product of Example 9 was solid bioactive glass. The product of Example 6 included not only the hollow bioactive glass but also the fine nanostructures because of low thermal degradation temperature of TEOS.

As mentioned above, the process of the present disclosure may shorten the process time so as able to apply to a continuous manufacturing process, and may precisely control the composition of the bioactive glass. Most importantly, the bioactive glass prepared by the process exhibits excellent bioactivity, in which hydroxyl apatite could be rapidly generated in half an hour. In addition, the hollow bioactive glass may be formed by using various calcining temperatures to save material cost, and can be used as a drug carrier. Therefore, the process is competitive in cost and product property and thus has extremely high commercial value.

Although the present disclosure has been described in considerable detail with reference to certain embodiments thereof, other embodiments are possible. Therefore, the spirit and scope of the appended claims should not be limited to the description of the embodiments contained herein.

It will be apparent to those ordinarily skilled in the art that various modifications and variations may be made to the structure of the present disclosure without departing from the scope or spirit of the disclosure, in view of the foregoing, it is intended that the present disclosure cover modifications and variations thereof provided they fall within the scope of the following claims.

What is claimed is:

1. A method for manufacturing bioactive glass, comprising:
mixing a precursor and a polar solvent to form a mixed solution, wherein the precursor includes a silicon precursor, a calcium precursor and a phosphorus precursor;
atomizing the mixed solution to form a mixture droplet; and
oxidizing the mixture droplet in an environment of 500° C. to 900° C. to form the bioactive glass.

2. The method of claim 1, wherein the polar solvent includes water, ethanol, acetone, formic acid, dimethylformamide (DMF) or a combination thereof.

3. The method of claim 1, wherein a molar ratio of the silicon precursor to sum of the calcium precursor and the phosphorus precursor is about 1:1 to about 9:1.

4. The method of claim 1, wherein a molar ratio of the calcium precursor to the phosphorus precursor is about 1:1 to about 3:1.

5. The method of claim 1, wherein mixing the precursor and the polar solvent comprises mixing a copolymer surfactant with the precursor and the polar solvent, the copolymer surfactant including ethylene oxide and propylene oxide and having an amount of about 10 to about 51 wt % based on the total weight of the precursor and the copolymer surfactant, and oxidizing the mixture droplet in the environment of 500° C. to 900° C. comprises forming mesoporous spherical bioactive glass.

6. The method of claim 5, wherein mixing the precursor and the polar solvent comprises mixing an acid with the precursor, the polar solvent, and the copolymer surfactant.

7. The method of claim 1, wherein the precursor has an amount less than or equal to 10 wt % based on the total weight of the precursor and the polar solvent.

8. A bioactive glass for forming hydroxyl apatite, which is manufactured by the method of claim 1.

9. The bioactive glass of claim 8, wherein bioactive glass has a ratio of silicon: sum of calcium and phosphorus in a range of about 1:1 to about 9:1.

10. The bioactive glass of claim 8, wherein the bioactive glass has a ratio of calcium: phosphorus in a range of about 1:1 to about 3:1.
